SEQUENCE LISTING (2 sequences)

SEQ ID NO: 1 - DNA, 2748 bp, Artificial Sequence
  chemical synthesized DNA
actccacacctggctggagcaggaaatgccgagcggcgcctgagccccagggaagcaggctaggatgtgagagacacagtcacctgcagcctaattactcaaaagctgtccccaggtcacagaagggagaggacatttcccactgaatctgtctgaaggacactaagccccacagctcaacacaaccaggagagaaagcgctgaggacgccacccaagcgcccagcaatggccctgcctggagaacatccaggctcagtgaggaagggtccagaagggaatgcttgccgactcgttggagaacaatgaaaaggaggaaactgtgactgaacctcaaaccccaaaccagcccgaggagaaccacattctcccagggacccagggcgggccgtgacccctgcggcggagaagccttggatatttccacttcagaagcctactggggaaggctgaggggtcccagctccccacgctggctgctgtgcagatgctggacgacagagccaggatggaggccgccaagaaggagaaggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgagagcagtggccgcaagcgcagggaggatgccacagccccacagcacccaggctccatggggaagtgctccccacacgtgctcgcagcctggcggggcaggaggcctggccttgtcaggacccaggccgcctgccataccgctggggaacagagcgggcctcttccctcagtttttcggtgggacagccccagggccctaacgggggtgcggcaggagcaggaacagagactctggaagccccccacctttctcgctggaatcaatttcccagaagggagttgctcactcaggactttgatgcatttccacactgtcagagctgttggcctcgcctgggcccaggctctgggaaggggtgccctctggatcctgctgtggcctcacttccctgggaactcatcctgtgtggggaggcagctccaacagcttgaccagacctagacctgggccaaaagggcagccaggggctgctcatcacccagtcctggccattttcttgcctgaggctcaagaggcccagggagcaatgggagggggctccatggaggaggtgtcccaagctttgaatacccccagagaccttttctctcccataccatcactgagtggcttgtgattctgggatggaccctcgcagcaggtgcaagagacagagcccccaagcctctgccccaaggggcccacaaaggggagaagggccagccctacatcttcagctcccatagcgctggctcaggaagaaaccccaagcagcattagcacaccccaagggacaaccccatcatatgacatgccaccctctccatgcccaacctaagattgtgtgggttttttaattaaaaatgttaaaagttttaaacatg

SEQ ID NO: 2 - DNA, 2801 bp, Artificial Sequence
  chemical synthesized DNA
cgctgcgttgccttaactccacacctggctggagcaggaaatgccgagcggcgcctgagccccagggaagcaggctaggatgtgagagacacagtcacctgcagcctaattactcaaaagctgtccccaggtcacagaagggagaggacatttcccactgaatctgtctgaaggacactaagccccacagctcaacacaaccaggagagaaagcgctgaggacgccacccaagcgcccagcaatggccctgcctggagaacatccaggctcagtgaggaagggtccagaagggaatgcttgccgactcgttggagaacaatgaaaaggaggaaactgtgactgaacctcaaaccccaaaccagcccgaggagaaccacattctcccagggacccagggcgggccgtgacccctgcggcggagaagccttggatatttccacttcagaagcctactggggaaggctgaggggtcccagctccccacgctggctgctgtgcagatgctggacgacagagccaggatggaggccgccaagaaggagaaggtagagcagatcctggcagagttccagctgcaggaggaggacctgaagaaggtgatgagacggatgcagaaggagatggaccgcggcctgaggctggagacccatgaagaggccagtgtgaagatgctgcccacctacgtgcgctccaccccagaaggctcagaagtcggggacttcctctccctggacctgggtggcactaacttcagggtgatgctggtgaaggtgggagaaggtgaggaggggcagtggagcgtgaagaccaaacaccagatgtactccatccccgaggacgccatgaccggcactgctgagatgctcttcgactacatctctgagtgcatctccgacttcctggacaagcatcagatgaaacacaagaagctgcccctgggcttcaccttctcctttcctgtgaggcacgaagacatcgataagggcatccttctcaactggaccaagggcttcaaggcctcaggagcagaagggaacaatgtcgtggggcttctgcgagacgctatcaaacggagaggggactttgaaatggatgtggtggcaatggtgaatgacacggtggccacgatgatctcctgctactacgaagaccatcagtgcgaggtcggcatgatcgtgggcacgggctgcaatgcctgctacatggaggagatgcagaatgtggagctggtggagggggacgagggccgcatgtgcgtcaataccgagtggggcgccttcggggactccggcgagctggacgagttcctgctggagtatgaccgcctggtggacgagagctctgcaaaccccggtcagcagctgtatgagaagctcataggtggcaagtacatgggcgagctggtgcggcttgtgctgctcaggctcgtggacgaaaacctgctcttccacggggaggcctccgagcagctgcgcacacgcggagccttcgagacgcgcttcgtgtcgcaggtggagagcgacacgggcgaccgcaagcagatctacaacatcctgagcacgctggggctgcgaccctcgaccaccgactgcgacatcgtgcgccgcgcctgcgagagcgtgtctacgcgcgctgcgcacatgtgctcggcggggctggcgggcgtcatcaaccgcatgcgcgagagccgcagcgaggacgtaatgcgcatcactgtgggcgtggatggctccgtgtacaagctgcaccccagcttcaaggagcggttccatgccagcgtgcgcaggctgacgcccagctgcgagatcaccttcatcgagtcggaggagggcagtggccggggcgcggccctggtctcggcggtggcctgtaagaaggcctgtatgctgggccagtgagagcagtggccgcaagcgcagggaggatgccacagccccacagcacccaggctccatggggaagtgctccccacacgtgctcgcagcctggcggggcaggaggcctggccttgtcaggacccaggccgcctgccataccgctggggaacagagcgggcctcttccctcagtttttcggtgggacagccccagggccctaacgggggtgcggcaggagcaggaacagagactctggaagccccccacctttctcgctggaatcaatttcccagaagggagttgctcactcaggactttgatgcatttccacactgtcagagctgttggcctcgcctgggcccaggctctgggaaggggtgccctctggatcctgctgtggcctcacttccctgggaactcatcctgtgtggggaggcagctccaacagcttgaccagacctagacctgggccaaaagggcagccaggggctgctcatcacccagtcctggccattttcttgcctgaggctcaagaggcccagggagcaatgggagggggctccatggaggaggtgtcccaagctttgaatacccccagagaccttttctctcccataccatcactgagtggcttgtgattctgggatggaccctcgcagcaggtgcaagagacagagcccccaagcctctgccccaaggggcccacaaaggggagaagggccagccctacatcttcagctcccatagcgctggctcaggaagaaaccccaagcagcattagcacaccccaagggacaaccccatcatatgacatgccaccctctccatgcccaacctaagattgtgtgggttttttaattaaaaatgttaaaagttttaaacatgctcgagatcggaaagaacatgtggatctacgggtggca

(19) United States
(12) Patent Application Publication
(10) Pub. No.: US 2021/0401910 A1
(43) Pub. Date: Dec. 30, 2021

(54) EXPRESSION VECTOR, PHARMACEUTICAL COMPOSITION AND METHOD FOR PREPARING THE SAME

(71) Applicant: REALITY BIOTECH COMPANY LIMITED, Queensway (HK)

(72) Inventor: Haidong HUANG, Beijing (CN)

(21) Appl. No.: 16/733,453

(22) Filed: Jan. 3, 2020

(51) Int. Cl. A61K 35/76; C12N 9/12; A61K 9/00; A61K 47/10; A61K 47/26; A61K 47/02

(52) U.S. Cl. CPC A61K 35/76; C12N 9/1205; C12Y 207/01002; A61K 9/0019; A61K 48/00; A61K 47/10; A61K 47/26; A61K 47/02; A61K 9/0073

(57) ABSTRACT

The present disclosure relates to the field of bio-pharmacy, in particular, to an expression and/or overexpression vector, a pharmaceutical composition and a method for preparing the same. The expression and/or overexpression vector of the present disclosure includes a human glucokinase mutant-encoding gene and an adeno-associated virus vector. The pharmaceutical composition of the present disclosure includes virus particles obtained by packaging the expression and/or overexpression vector. After the pharmaceutical composition of the present disclosure is injected into an animal, an obvious expression and/or overexpression of a glucokinase mutant may occur, which has a good blood glucose level lowering effect, and provides a powerful new approach for controlling/stabilizing blood glucose level and/or preventing and treating glucose metabolism disorders, especially for preventing and treating diabetes.

### TECHNICAL FIELD

The present disclosure relates to the field of bio-pharmacy, in particular, to an expression and/or overexpression vector, a pharmaceutical composition and a method for preparing the same.

### BACKGROUND

Glucokinase (GCK) is an important member of the hexokinase family, and its basic biological activity is to catalyze the phosphorylation of glucose. The human glucokinase-encoding gene is located on the short arm of chromosome 7 and has 10 exons. GCK consists of 465 amino acids, is specifically present in mature hepatocytes and islet β cells, and is involved in many important links in the process of glucose metabolism and insulin secretion.

Currently, it has been proved that the strength of insulin secretion response in the body is proportional to the metabolic rate of glucose in insulin β cells. Thus, enzymes that control the rate of glucose flowing into cells are considered to be the glucose sensors that regulate insulin release. GCK is a glucose sensor in islet β cells. When glucose enters β cells through transporter 2, it is phosphorylated under the action of glucokinase and enters the glycolytic pathway to produce ATP, the amount of ATP is proportional to the glucose entering β cells; and ATP can close the potassium ion channel on the β cell membrane, thereby causing depolarization, and further causing calcium influx and eventually resulting in insulin secretion. GCK activity is directly or indirectly regulated by the blood glucose level in the blood, thereby changing the metabolic rate of glucose in β cells and regulating insulin secretion. At the same time, the enzyme can regulate the blood glucose level by promoting the synthesis of hepatic glycogen and catalyzing the conversion of glucose into glucose 6-phosphate, so that abnormal GCK activity plays an important role in the development of glucose metabolism disorders.

Studies have found that in type 2 diabetes patients and some animal models, such as starved mice, high-fat diet-induced type 2 diabetes rats have significantly lower GCK activity in hepatocytes than in wild type rats; other research results also show that increasing GCK activity can significantly decrease blood glucose level, and current researches have confirmed that GCK gene mutations are closely related to the onset of maturity onset diabetes of the young (MODY), a subtype of type 2 diabetes, whose genetic mutations play a decisive role in the onset of MODY.

### SUMMARY

The technical problem to be solved by the present disclosure is to provide an expression and/or overexpression vector, a pharmaceutical composition and a method for preparing the same, in which the pharmaceutical composition can be used for controlling/stabilizing blood glucose level and/or preventing and treating diabetes.

The present disclosure provides an expression and/or overexpression vector, including a human glucokinase mutant-encoding gene and an adeno-associated virus vector.

Preferably, the human glucokinase mutant-encoding gene is:

(1) a nucleotide sequence shown in SEQ ID NO: 1; or

(2) a nucleotide sequence, which is same as the nucleotide sequence shown in SEQ ID NO: 1 except for an open reading frame at positions 487 to 1884 of the nucleotide sequence shown in SEQ ID NO: 1, and an open reading frame of which encodes an amino acid sequence same as that encoded by the open reading frame of the nucleotide sequence shown in SEQ ID NO: 1.

The present disclosure provides a pharmaceutical composition, including virus particles obtained by packaging the expression and/or overexpression vector according to the above technical solution.

Preferably, the pharmaceutical composition is in the form of an injection solution or an inhaler.

Preferably, the injection further includes a pharmaceutically acceptable excipient.

Preferably, the pharmaceutical composition further includes a protective agent and/or an osmotic pressure regulator, in which based on a content of the injection solution, a content of the protective agent is 0.01 to 30 wt %, and the protective agent is selected from one or more of inositol, sorbitol, and sucrose; and a content of the osmotic pressure regulator allows the osmotic pressure of the injection solution to be 200 to 700 mOsm/kg, and the osmotic pressure regulator is one or more of sodium chloride, potassium chloride, an organic halide and an organic halide derivative.

The present disclosure provides a method for preparing a pharmaceutical composition, including the following steps:

ligating a human glucokinase mutant-encoding gene and an adeno-associated virus vector to construct an expression and/or overexpression vector;

co-transfecting the expression and/or overexpression vector and a helper plasmid into a cell for virus packaging; and

collecting, concentrating and purifying a virus stock solution after culturing/passaging the cell to obtain virus particles.

Preferably, the cell is a human cell line with a complementary sequence necessary for replication of a defective virus of interest.

Preferably, the method further includes:

suspending the virus particles obtained with pharmaceutically acceptable excipients.

The present disclosure also provides the use of the pharmaceutical composition according to the above technical solution in the preparation of a medicament for preventing and treating diabetes.

As compared with the prior art, the present disclosure uses a human glucokinase mutant-encoding-gene and an adeno-associated virus vector to construct an expression and/or overexpression vector, and the virus particles obtained after packaging the expression vector can be used to form a pharmaceutical composition. After the pharmaceutical composition of the present disclosure is injected into an animal, an obvious expression and/or overexpression of the mutant glucokinase may occur, which has a significant blood glucose level lowering effect, and provides a powerful new approach for controlling/stabilizing blood glucose level and/or preventing and treating glucose metabolism disorders, especially for preventing and treating diabetes.

### BRIEF DESCRIPTION OF THE DRAWINGS

FIG. 1 is a schematic diagram showing the structure of adeno-associated virus vector PGMAAV-4895;

FIG. 2 is a schematic diagram showing the structure of an expression and/or overexpression vector carries a human glucokinase mutant-encoding gene;

FIG. 3 is a experimental result graph showing the GCK overexpression in the hepar of Balb/ca mice at 3, 4, and 5 weeks after virus injection;

FIG. 4 is a line chart showing the glycemia control effect on GK rats with different injection dose;

FIG. 5 is a line chart showing the glycemia control effects produced by the total averages of the control groups and the injection groups.

### DETAILED DESCRIPTION

In order to better understand the present disclosure, the preferable embodiments of the present disclosure will be described below in combination with Examples, but it should be understood that these descriptions are merely used to further illustrate the features and advantages of the present disclosure and are not intended to limit the present disclosure.

An embodiment of the present disclosure provides an expression and/or overexpression vector, which carries a human glucokinase mutant-encoding gene and an adeno-associated virus vector.

In the present disclosure, the human glucokinase mutant-encoding gene is:

(1) a nucleotide sequence shown in SEQ ID NO: 1; or

(2) a nucleotide sequence, which is same as the nucleotide sequence shown in SEQ ID NO: 1 except for an open reading frame at positions 487 to 1884 of the nucleotide sequence shown in SEQ ID NO: 1, and an open reading frame of which encodes an amino acid sequence same as that encoded by the open reading frame of the nucleotide sequence shown in SEQ ID NO: 1.

Preferably, the human glucokinase mutant-encoding gene has a nucleotide sequence shown in SEQ ID NO: 1.

The present disclosure uses an expression and/or overexpression vector formed by the adeno-associated virus vector and the human glucokinase mutant-encoding gene, and the expression and/or overexpression vector has the advantages of good expression effect and no adverse affect. As compared with the recombinant vector formed by the adenoviral vector, it has higher safety and fewer adverse affects.

The embodiment of the present disclosure provides a pharmaceutical composition, including virus particles obtained by packaging the expression and/or overexpression vector according to the above technical solution.

In the present disclosure, the virus particles obtained after packaging the expression and/or overexpression vector described in the above technical solution can be used directly as an active ingredient in a pharmaceutical composition, or used in combination with one or more pharmaceutically acceptable excipients as a pharmaceutical composition.

The pharmaceutically acceptable excipients refer to non-toxic solid, semi-solid or liquid fillers, diluents, encapsulating materials or other formulation excipients, for example, including, but not limited to, saline, buffered saline, glucose, water, glycerol, ethanol and the mixtures thereof. The pharmaceutical composition is suitable for intravenous, parenteral, sublingual, intracranial, intravaginal, intraperitoneal, intrarectal, intrabuccal or epidermal administration.

Optionally, the pharmaceutical composition is an injection solution or an inhaler.

Preferably, the injection further includes a pharmaceutically acceptable excipient.

The pharmaceutically acceptable excipient is a phosphate buffer with a pH value of 4.0 to 9.0; and 1 ml of the injection solution contains 10² to 10′⁷ copies of the human glucokinase mutant-encoding gene or 10² to 10¹⁷ copies of the recombinant vector.

Preferably, the pharmaceutical composition further includes a protective agent and/or an osmotic pressure regulator, in which based on a content of the injection solution, a content of the protective agent is 0.01 to 30 wt %, and the protective agent is selected from one or more of inositol, sorbitol, and sucrose; and a content of the osmotic pressure regulator allows the osmotic pressure of the injection solution to be 200 to 700 mOsm/kg, and the osmotic pressure regulator is one or more of sodium chloride, potassium chloride, an organic halide and an organic halide derivative.

The embodiment of the present disclosure also discloses a method for preparing a pharmaceutical composition, including the following steps:

ligating a human glucokinase mutant-encoding gene and an adeno-associated virus vector to construct an expression and/or overexpression vector;

co-transfecting the expression and/or overexpression vector and a helper plasmid into a cellline for virus packaging; and

collecting, concentrating and purifying a virus stock solution after culturing/passaging the cell to obtain virus particles.

The preparation method discloses a step of preparing virus particles. If the pharmaceutical composition further includes a pharmaceutically acceptable excipient, the obtained virus particles are suspended in pharmaceutically acceptable excipients according to the characteristics of the excipients and the virus particles to obtain a pharmaceutical composition.

According to the above method, the preparation process of the virus particles will be specifically explained.

Step 1, ligating a human glucokinase mutant-encoding gene and an adeno-associated virus vector to construct an expression and/or overexpression vector.

Specifically, a primer is designed to introduce the 5′ end of the primer into a homologous sequence of the end of the linearized cloning vector, so that the 5′ and 3′ end sequences of the amplified product are completely identical with the sequences at both ends of the linearized cloning vector, respectively;

The designed primers are used to amplify the human glucokinase mutant-encoding gene;

The adeno-associated virus vector is subjected to double-enzyme digestion;

The double-enzyme digested adeno-associated virus vector and the human glucokinase mutant-encoding gene are subjected to ligation;

After transformation and screening, an expression and/or overexpression vector is constructed.

Preferably, the constructed expression and/or overexpression vector is sequenced to identify whether the sequence of the insert fragment in the clone is completely identical to the sequence of the human glucokinase mutant-encoding gene.

Step 2, cotransfecting the expression and/or overexpression vector and a helper plasmid into a cell for virus packing.

Preferably, the cell is a human cell line with a complementary sequence necessary for replication of a defective virus of interest, and more preferably an AAV-293 cell.

The process specifically includes:

preparing an AAV-293 cell; and

cotransfecting the expression and/or overexpression vector and a helper plasmid into an AAV-293 cell for virus packaging.

Step 3, collecting, concentrating and purifying a virus stock solution after culturing/passaging the cell to produce virus particles.

The process specifically includes:

The transfected AAV-293 is cultured, and the cells and cell supernatants are collected; and the cells and cell supernatants are virus stock solution;

The collected virus stock solution is concentrated and purified to obtain virus particles.

The virus particles are generally packed and stored in the form of a virus concentrate.

The embodiment of the present disclosure also provides the use of the pharmaceutical composition according to the above technical solution in the preparation of a medicament for controlling/stabilizing blood glucose level and/or preventing and treating diabetes.

In order to further understand the present disclosure, the expression and/or overexpression vector and pharmaceutical composition provided by the present disclosure will be described in detail in conjunction with the following Examples, but the protection scope of the present disclosure is not limited by the following Examples.

### Example 1 Construction of Expression and/or Overexpression Vector

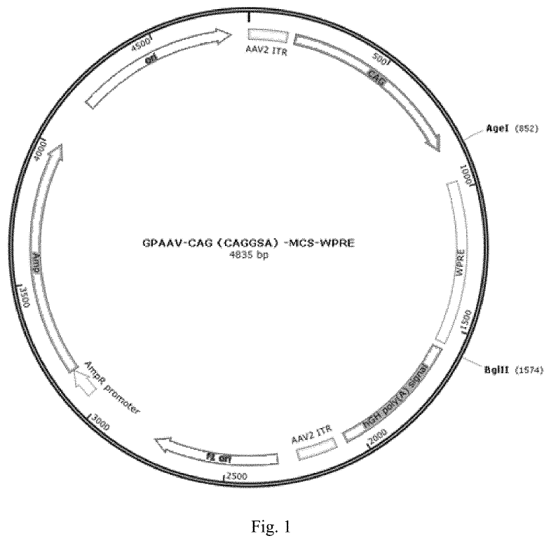

(1) Adeno-associated virus vector was PGMAAV-4895, and its structure was shown in FIG. 1.

(2) The primer for PCR amplification fragment was designed to introduce the 5′ end of the primer into a homologous sequence of the end of the linearized adeno-associated virus vector, so that the 5′ and 3′ end sequences of the amplification product were completely identical with the sequences at both ends of the linearized adeno-associated virus vector, respectively.

The designed primers were Primer-F and Primer-R;

The sequence of Primer-F was:

CGCTGCGTTGCCTTAACTCCACACCTGGCTGGAGC;

The sequence of Primer-R was:

TGCCACCCGTAGATCCACATGTTCTTTCCGATCTCGAGC.

(3) Double Enzyme Digestion of the Adeno-Associated Virus Vector

The bacterial suspension solution containing the vector plasmid was cultured/passaged overnight, and the plasmid was extracted from 3 to 5 ml of fresh bacterial suspension. For specific methods, please refer to the instructions of QIAGEN Plasmid Mini Kit.

1 μg of fresh plasmid is double-enzyme digested with the corresponding restriction enzymes. The enzyme digestion system was shown as follows:

| | | |
|---|---|---|
| Vector | 1 | μg |
| green Buffer | 3 | μL |
| AgeI | 1.5 | μL |
| BglII | 1.5 | μL |

| | |
|---|---|
| ddH₂O | Up to 30 μL |

and enzyme digestion was performed at 37° C. for about 3 h.

The enzyme-digested product was subjected to agarose gel electrophoresis. After the electrophoresis was completed, the gel was recovered as follows: the gel strip containing the target fragment was cut under a UV lamp. The weight of the gel was calculated by subtracting the weight of the empty tube from the total weight measured by a balance, in which the volume of the gel was calculated as 100 mg=100 and 1 gel volume of binging solution was added and placed in a water bath at 65° C. to thoroughly melt the gel. The EP tube was shaken appropriately during this period to speed up the dissolution of the gel.

All the above liquid was transferred into the filter column and centrifuged at 13,000 rpm for 30 s (which may be repeated once). Then, the liquid in the tube was discarded, and 500 μL of WA solution was added into the column and centrifuged at 13000 rpm for 30 s. The liquid in the tube was discarded, and then 500 μL of wash solution was added into the column and centrifuged at 13,000 rpm for 30 s (which may be repeated once). It was then centrifuged for 3 min. A filter column was placed in a new 1.5 mL EP tube and dried at room temperature. Finally, 35 μL of ddH₂O was added into the column, stood for 5 min, and was centrifuged at 13,000 rpm for 1.5 min. In order to increase recovery rate, the lysed DNA can be added to the column and centrifuged for 1 min. The column was discarded, that was, the adeno-associated virus vector fragment was recovered, and the concentration was measured.

(4) Amplification of the Target Fragment

The synthesized primer was diluted into a stock solution with a final concentration of 10 μmon.

PCR amplification was performed using the diluted primers and the nucleotide sequence shown in SEQ ID NO: 1. The system was shown as follows:

| | |
|---|---|
| Template | 1 to 2 μg |
| Primer-F | 2 μL |
| Primer-R | 2 μL |
| PCR mix | 25 μL |
| ddH₂O | Up to 50 μL |

The above materials were added into the thin-wall tube and mixed, and then placed them into the PCR instrument. An appropriate annealing temperature and extension temperature were selected, and then the PCR amplification was started.

After PCR, agarose gel electrophoresis was performed, and the gene of interest was recovered. The recovery method was the same as above.

(5) Ligation of Adeno-Associated Virus Vector and the Target Fragment

The concentration of the recovered vector and the target fragment were measured;

The amount of the optimum cloning vector in Hieff Clone™ recombination reaction system was 0.01-0.3 pmol; and the molar ratio of optimum cloning vector to inserted fragment was 1:2, that was, the amount of optimum inserted fragment was 0.01-0.6 pmol. The DNA mass corresponding to these moles can be roughly calculated from the following formula:

Amount of optimum cloning vector=[0.02×number of base pairs in cloning vector] ng (0.01-0.3 pmol)

Amount of optimum inserted fragment=[0.04×number of inserted base pairs] ng (0.01-0.6 pmol)

Ligation of adeno-associated virus vector and the target fragment, and the target fragment was shown as follows:

| | |
|---|---|
| ddH₂O | Up to 20 μl |
| 5 × CE Buffer | 4 μl |
| Adeno-associated virus vector | 50 to 200 ng |
| Insert fragment amplification | 20 to 200 ng |
| products | |
| Recombinase | 2 μl |

Ligation was performed at 37 degrees for 30 minutes.

(6) Transformation

Competent cells were placed on ice (4° C.) and allow them to thaw naturally. 10 μl of the ligation product were added to competent cells and placed on ice (4° C.) for 30 min.

Then, they were placed into water bath at 42° C. for heat shocking for 90 s. Then, they were quickly placed on ice (4° C.) for 2 to 3 min.

500 μL of antibiotic-free SOC medium were added and incubated at 37° C. for 45 min under shaking at 225 rpm.

They were centrifuged at 3000 rpm for 2 min, and 900 μL of the supernatant was discarded. The bacterial precipitation at the bottom of the tube was blown apart, added to the culture plate containing an antibiotic (ampicillin or kanamycin etc.) according to the resistance which was encoded by the vector, coated evenly by a sterilized applicator (the temperature of the applicator should not be too high, so as to avoid scalding the bacterial), and placed in an incubator at 37° C. overnight for incubation.

Two clones were selected from each clone for sequencing identification.

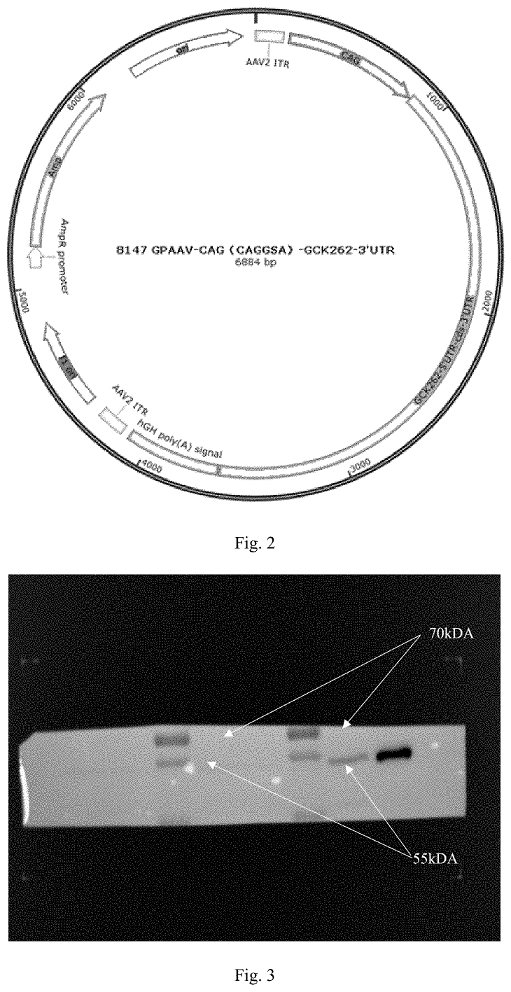

The structure of the constructed expression and/or overexpression vector was shown in FIG. 2.

After sequencing, in the recombinant clone, the inserted sequence was shown in SEQ ID NO: 2.

After comparison, the sequence of the inserted fragment in the recombinant clone was completely consistent with the sequence of the target fragment SEQ ID NO: 1, so the recombinant expression and/or overexpression vector was successfully constructed.

### Example 2 Packaging of the Adeno-Associated Virus

(1) AAV 293 cell culture/passage

One day before the transfection, the grown cells were passed into a 10 cm culture dish at an appropriate ratio, and prepared for transfection when the cells grow to 70% to 80%.

(2) discard used medium and replace with fresh prepared medium before transfection

The cells to be transfected were replaced with fresh medium at 1 to 2 hours before transfection.

(3) Transfection

Apple a sterile 1.5 ml EP tube or 15 ml centrifuge tube, the transfection mixture was shown as follows:

| | | |
|---|---|---|
| DMEM | 1 | ml |
| Expression plasmid | 6 | μg |
| Helper plasmid (pay attention to | 18 | μg |
| different serotypes) | | |
| HG transgenic reagent | 72 | μl |

The mixture was placed at room temperature for 15 to 20 min, added dropwise uniformly to a petri dish that has been replaced in advance, and then placed into a CO₂ incubator for cultivation.

(4) Addition of Enhancement Buffer

10 to 12 hours after the transfection, 100×enhancement buffer was added dropwise uniformly to promote transfection at 120 μl/dish.

(5) Medium Refresh

18 to 20 hours after the transfection, the medium was carefully sucked and discarded in a waste liquid cup containing disinfectant, and then fresh medium was added.

(6) Virus Collection

72 hours after the transfection, cells and cell supernatants were collected for subsequent concentration and purification.

(7) Virus Concentration and Purification

The cell suspension was transferred in a dry ice ethanol bath and a water bath at 37° C. repeatedly, and freeze-thawed four times. It was Centrifuged at 4000 rpm for 10 min to collect supernatant

Concentration of AAV

The virus supernatant was loaded into an ultra-centrifuge tube, and centrifuged at 100,000 g for 2 h.

Benzonase was added at 37° C. for 20 min.

It was filtered through a 0.22 um filter membrane, and the supernatant was transferred to a horizontal ultra-centrifuge tube.

Purification of AAV

60%, 40%, 25%, and 15% of iodixanol were prepared in a gradient order of 60% iodixanol, 40% iodixanol, 25% iodixanol, and 15% iodixanol.

Centrifuged at 270,000 g, 4 degrees, for 2 h.

It was transferred to a 50 kD ultra-filtration tube and centrifuged at 4000 rpm for 15 min, and the precipitation was discarded.

(8) Subpackage and Storage of Virus

Sub-pack the virus suspension and stored at −80 degree.

(9) Determination of Virus Titer

The quantification of the current determined AAV was performed by the quantitative PCR detection of the number of genomic copies of the AAV vector in the genome, so as to determine the number of AAV virus particles.

Quantitative PCR Experiment Steps

Prepare Samples and standards, in which standard plasmids and test samples were diluted to the original concentration of 10⁻², 10⁻⁴, 10⁻⁵, 10⁻⁶, and 10⁻⁷;

The total volume of the reaction tube was calculated according to the number of reactions (X) (to prepare two replicate wells per gradient and to prepare one more for every 10 reactions):

Add 18 μl of reaction solution to each reaction well then add 2 μl of template;

The volume of each component in the PCR system was shown as follows:

| Component | Volume | |
|---|---|---|
| Ultrapure water | 7 | ul |
| 2 × SYBR Mix | 10 | ul |
| Upstream primer (10 uM) | 0.5 | ul |
| Downstream primer | 0.5 | ul |
| (10 uM) | | |
| Template | 2 | ul |
| Total system | 20 | ul |

The annealing temperature was set to 60° C., and the Ct value was obtained according to standard operations and the number of copies in the AAV sample was calculated.

The experimental results were shown in Table 1:

TABLE 1

| | | | Titer | |
|---|---|---|---|---|
| | | Dilution | after | Average |
| Sample name | Titer | factor | dilution | value |
| 8147 (GPAAV-CAG (CAGGSA)- | 3.09E+10 | 1.00E+02 | 3.09E+12 | 3.09E+12 |
| GCK262-3′UTR) -2 | | | | |
| 8147 (GPAAV-CAG (CAGGSA)- | 3.13E+08 | 1.00E+04 | 3.13E+12 | 3.11E+12 |
| GCK262-3′UTR) -4 | | | | |
| 8147 (GPAAV-CAG (CAGGSA)- | 3.03E+07 | 1.00E+05 | 3.03E+12 | 3.08E+12 |
| GCK262-3′UTR) -5 | | | | |
| 8147 (GPAAV-CAG (CAGGSA)- | 3.14E+06 | 1.00E+06 | 3.14E+12 | 3.10E+12 |
| GCK262-3′UTR) -6 | | | | |
| 8147 (GPAAV-CAG (CAGGSA)- | 3.05E+05 | 1.00E+07 | 3.05E+12 | 3.09E+12 |
| GCK262-3′UTR) -7 | | | | |
| 10547 (GPAAV-CAG (CAGGSA)- | 3.08E+10 | 1.00E+02 | 3.08E+12 | 3.08E+12 |
| GCK262-3′UTR-WPRE) -2 | | | | |
| 10547 (GPAAV-CAG (CAGGSA)- | 3.16E+08 | 1.00E+04 | 3.16E+12 | 3.12E+12 |
| GCK262-3′UTR-WPRE) -4 | | | | |
| 10547 (GPAAV-CAG (CAGGSA)- | 3.14E+07 | 1.00E+05 | 3.14E+12 | 3.13E+12 |
| GCK262-3′UTR-WPRE) -5 | | | | |
| 10547 (GPAAV-CAG (CAGGSA)- | 3.04E+06 | 1.00E+06 | 3.04E+12 | 3.10E+12 |
| GCK262-3′UTR-WPRE) -6 | | | | |
| 10547 (GPAAV-CAG (CAGGSA)- | 3.07E+05 | 1.00E+07 | 3.07E+12 | 3.10E+12 |
| GCK262-3′UTR-WPRE) -7 | | | | |

Upon the detection: the titer of 8147 (GPAAV-CAG (CAGGSA)-GCK262-3′UTR) was 3.09E+12 VG/mL.

The titer of 10547 (GPAAV-CAG (CAGGSA)-GCK262-3′UTR-WPRE) was 3.10E+12 VG/mL.

### Example 3 Experimental Effect of GCK Protein Expression In Vivo

(1) Balb/ca mice were used to measure the duration of adeno-associated virus-mediated exogenous protein expression after injection in vivo

The virus concentrate prepared in Example 2 was prepared into an injection, and the highest concentration was 1E+12 VG/ml.

The safe volume of tail vein injection that can be acceptable to Balb/ca mice was 200 ul. From this calculation, the adeno-associated virus dose received by the mice was 2E+11 vg/animal through the conversion of the mouse body weight (25 g/mouse),

2E+11vg÷0.025 (kg)=8E+12vg/kg of body weight

Balb/ca mice were randomly divided into 3 injection groups, 20 mice each group with an injection dose, 8E+12 (Vg/kg of body weight). A corresponding control group was set for each injection group. Mice were sacrificed at 3 weeks 4 weeks and 5 weeks after infection to obtain liver to check the GCK overexpression by western blot assay.

The results of western blot assay were shown in FIG. 3.

In the electrophoresis diagram of FIG. 3, from left to right:

liver of control group at 3 weeks, liver of injection group at 3 weeks, molecular weight marker (ladder), liver of control at 4 weeks, liver of injection group at 4 weeks, molecular weight marker (ladder), liver of control group at 5 weeks, liver of injection group at 5 weeks.

As can be seen from FIG. 3, the expression and/or overexpression of adeno-associated virus-mediated exogenous protein occurs at the fifth week after the animal receiving the injection, suggesting that the onset time of the adeno-associated virus preparation should start in the fifth week after the injection.

(2) GK rats were used to verify the effect of adeno-associated virus injection on glycemia control in vivo.

GK rats: Spontaneous type 2 diabetes Goto-Kakizaki (GK) rats were non-obese type 2 diabetic rats formed by repeatedly selecting the inbreeding Wistar rats, in which a large number of biological characteristics reflected in their growth and development were highly similar to those of human type 2 diabetes.

the experimental design shown in Table 2 was performed as follows:

TABLE 2

| | | | | | Dosage of | |
|---|---|---|---|---|---|---|
| | | Number | | | administration | Time and |
| | Method of | of | | Times of | (VG/Kg | method of |
| Group | administration | animal | Group | administration | weight) | detection |
| Blank | N/A | 11 | Blank | N/A | N/A | Blood glucose level |
| control | | | control | | | was tested once a |
| Dose | Tail intravenous | 6 | Dose | Single dose | 1E+11 | week at 2 weeks after |
| Group 1 | injection | | Group 1 | | | injection and blood |
| Dose | Tail intravenous | 7 | Dose | Single dose | 2E+11 | glucose level was |
| Group 2 | injection | | Group 2 | | | tested twice a week |
| Dose | Tail intravenous | 6 | Dose | Single dose | 5E+11 | at 5 weeks. Tail vein |
| Group 3 | injection | | Group 3 | | | blood was collected, |
| Dose | Tail intravenous | 6 | Dose | Single dose | 1E+12 | and tested for blood |
| Group 4 | injection | | Group 4 | | | glucose level |
| Dose | Tail intravenous | 6 | Dose | Single dose | 2E+12 | measurement by using |
| Group 5 | injection | | Group 5 | | | Roche blood glucose |
| Dose | Tail intravenous | 6 | Dose | Single dose | 5E+12 | level meter and blood |
| Group 6 | injection | | Group 6 | | | glucose level test strip. |

The experimental results of the control group were shown in Table 3 below:

TABLE 3

| | Result of blood glucose test, blood glucose level unit: mmol/L | | | | | | | | | | |
|---|---|---|---|---|---|---|---|---|---|---|---|
| | 20190620 | | | | | | | | | | |
| | Initial | | | | | | | | | | |
| | value of | | | | | | | | | | |
| No. | injection | 20190701 | 20190708 | 20190711 | 20190716 | 20190722 | 20190724 | 20190726 | 20190729 | 20190801 | 20190806 |
| Control | 11.0 | 9.0 | 12.6 | 9.9 | 9.4 | 15.1 | 12.8 | 11.4 | 13.1 | 14.4 | 12.0 |
| Group | 22.5 | 18.4 | 21.0 | 20.0 | 16.6 | 18.7 | 23.4 | 24.4 | 26.8 | 24.0 | 23.4 |
| | 13.9 | 12.9 | 13.5 | 13.7 | 14 | 18.5 | 21.5 | 22.0 | 21.4 | 19.9 | 16.7 |
| | 8.8 | 12.3 | 16.1 | 10.0 | 8.4 | 14.9 | 12.2 | 12.5 | 16.9 | 10.8 | 15.2 |
| | 9.1 | 12.0 | 17.4 | 7.9 | 6.5 | 15.8 | 13.8 | 13.8 | 15.0 | 15.3 | 15.3 |
| | 17.6 | 14.9 | 18.1 | 16.3 | 9.3 | 16.3 | 15.6 | 16.6 | 17.4 | 14.2 | 17.3 |
| | 13.1 | 14.5 | 19.2 | 19.6 | 12 | 16.6 | 20.5 | 22.3 | 20.5 | 16.1 | 18.0 |
| | 16.4 | 15.1 | 12.3 | 13.8 | 10 | 14.2 | 16.8 | 17.5 | 18.5 | 20.0 | 18.5 |
| | 12.5 | 16.8 | 15.6 | 13.7 | 15.4 | 17.8 | 19.6 | 18.6 | 19.8 | 21.0 | 18.6 |
| | 10.0 | 9.0 | 16.8 | 7.2 | 13.9 | 16.5 | 16.1 | 20.2 | 18.7 | 19.2 | 20.0 |
| | 14.8 | 13.7 | 14.4 | 20.8 | 23 | 19.1 | 20 | 20.1 | 20.5 | 17.9 | 22.6 |

The results of blood glucose level test of dose groups 1, 2 and 3 of GK rats were shown in Table 4.

TABLE 4

| | Result of blood glucose level test, blood glucose level unit: mmol/L | | | | | | | | | | |
|---|---|---|---|---|---|---|---|---|---|---|---|
| | 20190620 | | | | | | | | | | |
| | Initial | | | | | | | | | | |
| | value of | | | | | | | | | | |
| No. | injection | 20190701 | 20190708 | 20190711 | 20190716 | 20190722 | 20190724 | 20190726 | 20190729 | 20190801 | 20190806 |
| Dose | 9.9 | 7.7 | 15.6 | 7.2 | 13.9 | 10.5 | 9.1 | 8.3 | 7.1 | 10.4 | 9.6 |
| Group 1 | 10.0 | 7.4 | 9.7 | 9.0 | 11.0 | 12.0 | 8.9 | 9.2 | 8.2 | 9.4 | 9.8 |
| | 12.9 | 6.8 | 8.4 | 6.6 | 8.8 | 8.6 | 8.1 | 6.8 | 7.3 | 10.2 | 9.7 |
| | 20.4 | 17.9 | 19.4 | 19.6 | 12.0 | 12.9 | 9.4 | 7.2 | 7.8 | 9.3 | 8.1 |
| | 13.4 | 10.6 | 15.8 | 9.7 | 7.2 | 11.4 | 6.2 | 13.2 | 10.0 | 9.7 | 8.3 |
| | 14.0 | 15.2 | 16.4 | 11.3 | 6.2 | 13.0 | 16.0 | 9.3 | 10.0 | 10.3 | 10.9 |
| Dose | 20.0 | 11.4 | 14.2 | 9.9 | 7.7 | 10.2 | 10.3 | 11.9 | 7.4 | 11.4 | 10.2 |
| Group 2 | 8.9 | 15.2 | 16.3 | 8.7 | 7.0 | 9.4 | 10.2 | 9.0 | 8.9 | 11.8 | 12.8 |
| | 10.4 | 20.7 | 24.2 | 22.2 | 18.6 | 15.4 | 12.6 | 19.0 | 9.6 | 10.8 | 10.1 |
| | 7.0 | 7.5 | 10.3 | 8.1 | 6.5 | 9.6 | 8.8 | 9.3 | 8.7 | 8.2 | 6.1 |
| | 10.6 | 9.4 | 11.4 | 13.8 | 10.0 | 8.6 | 9.6 | 9.1 | 10.8 | 8.3 | 10.7 |
| | 18.5 | 15.1 | 15.0 | 15.4 | 10.4 | 10.8 | 10.9 | 7.5 | 8.4 | 9.8 | 9.1 |
| | 8.7 | 7.7 | 7.7 | 8.9 | 7.3 | 7.6 | 8.7 | 7.3 | 8.9 | 10.2 | 9.7 |
| Dose | 11.0 | 8.0 | 7.5 | 7.0 | 7.2 | 5.9 | 6.2 | 6.4 | 6.8 | 5.8 | 5.8 |
| Group 3 | 10.1 | 10.0 | 14.7 | 13.7 | 13.7 | 15.8 | 13.0 | 8.5 | 9.5 | 9.8 | 10.2 |
| | 11.0 | 9.0 | 10.4 | 8.4 | 11.0 | 12.5 | 10.7 | 10.0 | 8.2 | 10.2 | 12.8 |
| | 20.3 | 11.4 | 22.5 | 21.5 | 14.8 | 10.7 | 8.4 | 7.5 | 12.0 | 11.5 | 8.6 |
| | 11.8 | 10.9 | 15.0 | 9.0 | 8.4 | 11.7 | 9.7 | 8.4 | 9.3 | 9.4 | 8.3 |
| | 12.5 | 10.2 | 14.6 | 7.7 | 10.3 | 7.5 | 11.2 | 12.4 | 10.1 | 10.8 | 9.9 |

The results of blood glucose level test of dose groups 4, 5, and 6 of GK rats were shown in Table 5.

TABLE 5

| | Result of blood glucose level test, blood glucose level unit: mmol/L | | | | | | | | | | |
|---|---|---|---|---|---|---|---|---|---|---|---|
| | 20190620 | | | | | | | | | | |
| | Initial value of | | | | | | | | | | |
| No. | injection | 20190701 | 20190708 | 20190711 | 20190716 | 20190722 | 20190724 | 20190726 | 20190729 | 20190801 | 20190806 |
| Dose | 16.1 | 18.0 | 21.5 | 13.7 | 15.4 | 9.1 | 8.4 | 9.2 | 8.9 | 10.5 | 11.3 |
| Group 4 | 12.8 | 17.7 | 7.7 | 11.5 | 12.9 | 10.0 | 9.7 | 8.4 | 8.8 | 9.8 | 9.6 |
| | 7.7 | 8.5 | 8.3 | 6.7 | 7.2 | 7.9 | 8.2 | 9.0 | 8.1 | 6.0 | 6.1 |
| | 28.0 | 16.6 | 20.2 | 20.7 | 11.2 | 10.4 | 9.1 | 9.1 | 8.3 | 10.0 | 11.7 |
| | 8.9 | 11.4 | 17.0 | 8.3 | 7.5 | 7.2 | 8.4 | 8.6 | 9.3 | 9.7 | 11.0 |
| | 11.4 | 11.9 | 7.3 | 9.3 | 5.0 | 9.6 | 8.7 | 7.9 | 8.8 | 9.1 | 11.3 |
| Dose | 14.2 | 17.5 | 22.4 | 15.8 | 11.4 | 10.7 | 9.1 | 10 | 8.0 | 10.9 | 10.5 |
| Group 5 | 12.5 | 11.7 | 10.7 | 11.8 | 7.8 | 9.3 | 8.5 | 8.2 | 7.7 | 9.8 | 10.7 |
| | 8.8 | 13.2 | 12.1 | 11.3 | 7.2 | 10.5 | 7.4 | 7.9 | 9.6 | 10.1 | 10.8 |
| | 10.5 | 14.2 | 18.8 | 11.1 | 17.1 | 10.7 | 9.9 | 7.4 | 8.3 | 10.0 | 10.8 |
| | 15.6 | 14.5 | 16.6 | 15.3 | 20.4 | 15.2 | 10.9 | 8.4 | 8.9 | 10.3 | 11.4 |
| | 19.2 | 19.9 | 16.6 | 18.7 | 20.9 | 15.6 | 12.5 | 10.6 | 9.4 | 10.0 | 9.7 |
| Dose | 19.4 | 21.6 | 8.0 | 20.8 | 23.0 | 10.5 | 7.8 | 8.6 | 7.8 | 9.1 | 10.1 |
| Group 6 | 10.8 | 7.2 | 12.9 | 6.8 | 6.5 | 7.8 | 11.8 | 7.9 | 8.6 | 8.2 | 8.4 |
| | 9.6 | 9.8 | 16.8 | 6.7 | 6.5 | 8.0 | 7.2 | 6.9 | 9.4 | 7.2 | 6.5 |
| | 9.1 | 13.7 | 17.9 | 14.9 | 11.8 | 10.4 | 6.0 | 7.4 | 8.2 | 10.9 | 11.7 |
| | 7.3 | 19.3 | 14.5 | 10.2 | 7.9 | 12.8 | 13.3 | 7.4 | 10.4 | 11.2 | 10.1 |
| | 22.5 | 8.4 | 20.4 | 19.1 | 20.5 | 14.3 | 12.4 | 7.6 | 8.0 | 10.4 | 8.2 |

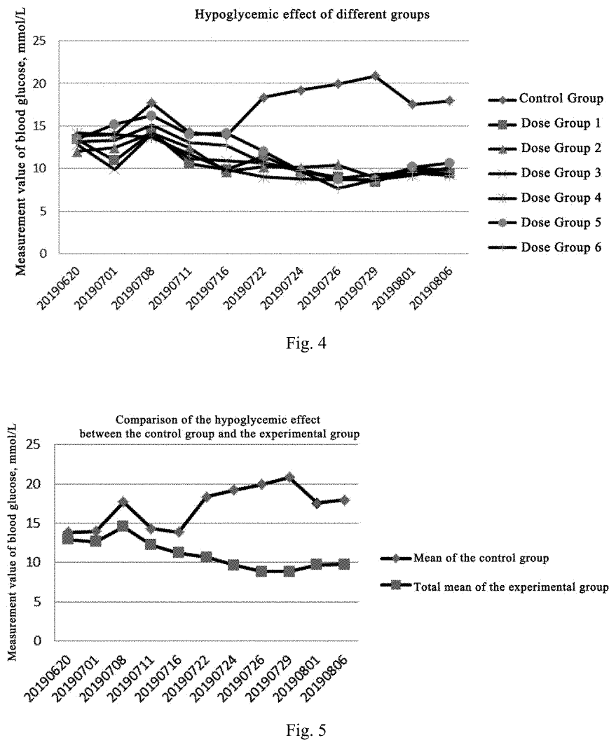

The contents of Tables 3 to 5 were drafted into line charts, as shown in FIG. 4.

Based on the results in Tables 3 to 5, the total average value of the injection group was calculated, and a line chart was made with the control group according to the measurement time, as shown in FIG. 5.

The experimental results show that each dose group can produce a glycemic control/stabilization effect on GK rats (FIG. 4), and there was no significant difference in glycemic control/stabilization effect among different dose groups. Thus, within the gradient range of this experiment, the adeno-associated virus failed to show a dose-benefit ratio relationship. There were two possible reasons:

The adeno-associated virus-mediated exogenous expression and/or overexpression in vivo has no obvious linear relationship with the amount of infection;

Due to the limitation of the concentration of adeno-associated virus prepared in the laboratory, the span of the dose concentration in this experiment had not yet reached a certain linear segment of the dose-benefit ratio relationship, and thus failed to show its inherent dose-benefit ratio characteristics.

The injection group and the control group showed a significant difference in the overall average blood glucose level (FIG. 5), and the overall blood glucose level decreased by about 30%.

Adeno-associated virus-mediated exogenous expression and/or overexpression experiments showed that strong expression and/or overexpression of exogenous proteins occurred at five weeks after the injection. As can be seen from FIG. 5, the blood glucose level fluctuations in the control group and the injection group were similar from 1 to 4 weeks after the injection; and at the fifth week, the blood glucose level of the control group continued to increase, while the blood glucose level of the injection group decreased, and its blood glucose level average fluctuates within the range of 7 to 10 mmol/L along with time.

The description of the above Examples is merely used for helping to understand the method according to the present disclosure and its core idea. It should be noted that a person skilled in the art may make further improvements and modifications to the disclosure without departing from the principle/spirit of the present disclosure, and these improvements and modifications shall also fall within the scope of the present disclosure.

The above description of the disclosed Examples allows one skilled in the art to implement or use the present disclosure. Various modifications to these Examples would be apparent to one skilled in the art, and the general principles defined herein may be applied to other Examples without departing from the spirit or scope of the disclosure. Therefore, the present disclosure will not be limited to the Examples shown herein, but should conform to the widest scope consistent with the principles and novel features disclosed herein.

1. An expression and/or overexpression vector, comprising a human glucokinase mutant-encoding gene and an adeno-associated virus vector, wherein the adeno-associated virus vector is PGMAAV-4895.

2. The expression and/or overexpression vector of claim 1, wherein the human glucokinase mutant-encoding gene is:
(1) a nucleotide sequence shown in SEQ ID NO: 1; or
(2) a nucleotide sequence, which is same as the nucleotide sequence shown in SEQ ID NO: 1 except for an open reading frame at positions 487 to 1884 of the nucleotide sequence shown in SEQ ID NO: 1, and an open reading frame of the nucleotide sequence encodes an amino acid sequence same as that encoded by the open reading frame of the nucleotide sequence shown in SEQ ID NO: 1.

3. A pharmaceutical composition, comprising virus particles obtained by packaging the expression and/or overexpression vector of claim 1.

4. The pharmaceutical composition of claim 3, wherein the pharmaceutical composition is in the form of an injection solution or an inhaler.

5. The pharmaceutical composition of claim 4, wherein the injection further comprises a pharmaceutically acceptable excipient.

6. The pharmaceutical composition of claim 5, further comprising a protective agent and/or an osmotic pressure regulator, wherein based on a content of the injection solution, a content of the protective agent is 0.01 to 30 wt %, and the protective agent is selected from one or more of inositol, sorbitol, and sucrose; and a content of the osmotic pressure regulator allows the osmotic pressure of the injection solution to be 200 to 700 mOsm/kg, and the osmotic pressure regulator is one or more of sodium chloride, potassium chloride, an organic halide and an organic halide derivative.

7. A method for preparing a pharmaceutical composition, comprising:
ligating a human glucokinase mutant-encoding gene and an adeno-associated virus vector to construct an expression and/or overexpression vector;
cotransfecting the expression and/or overexpression vector and a helper plasmid into a cell for virus packaging; and
collecting, concentrating and purifying a virus stock solution after culturing/passaging the cell to obtain virus particles.

8. The method of claim 7, wherein the cell is a human cell line with a complementary sequence necessary for replication of a defective virus of interest.

9. The method of claim 7, further comprising:
mixing the virus particles obtained with pharmaceutically acceptable excipients.

10. A method of using the pharmaceutical composition of claim 3 to control/stabilize blood glucose level and/or prevent and treat diabetes.

11. The expression and/or overexpression vector of claim 1, wherein the adeno-associated virus vector has designed Primer-F and Primer-R, and
wherein the Primer-F comprises a sequence of:
CGCTGCGTTGCCTTAACTCCACACCTGGCTGGAGC;
the Primer-R comprises a sequence of:
TGCCACCCGTAGATCCACATGTTCTTTCCGATCTCGAGC.
